(19) United States
(12) Patent Application Publication
(10) Pub. No.: US 2010/0063500 A1
(43) Pub. Date: Mar. 11, 2010

(54) Apparatus, System and Method for Performing an Electrosurgical Procedure

(75) Inventor: Melissa J. Muszala, Boulder, CO (US)

Correspondence Address: TYCO Healthcare Group LP;Attn: IP Legal, 5920 Longbow Drive, Mail Stop A36, Boulder, CO, 80301-3299, US

(73) Assignee: TYCO Healthcare Group LP

(21) Appl. No.: 12/204,976

(22) Filed: Sep. 5, 2008

(51) Int. Cl. A61B 18/14; A61B 18/12

(52) U.S. Cl. 606/48; 606/52; 606/51

(57) ABSTRACT

An electrosurgical apparatus that includes a housing having at least one shaft extending therefrom that operatively supports an end effector assembly at a distal end thereof is provided. The end effector assembly includes first and second jaw members pivotably connected to each other and moveable from an open spaced apart position to a closed position. Each of the jaw members operatively couples to an electrically conductive seal plate. One or both of the jaw members is configured to support one or more filaments thereon for selectively sectioning tissue. The electrically conductive seal plates and the filament are adapted to connect to an electrical surgical energy source. The electrosurgical apparatus is in operative communication with a control system having one or more control algorithms for independently controlling and monitoring the delivery of electrosurgical energy from the source of electrosurgical energy to the one or more filaments and the tissue sealing plate.

### BACKGROUND

1. Technical Field

The following disclosure relates to an apparatus, system, and method for performing an electrosurgical procedure and, more particularly, to an apparatus, system and method that utilizes energy based sectioning to cut and/or section tissue as required by an electrosurgical procedure.

2. Description of Related Art

It is well known in the art that electrosurgical generators are employed by surgeons in conjunction with electrosurgical instruments to perform a variety of electrosurgical surgical procedures (e.g., tonsillectomy, adenoidectomy, etc.). An electrosurgical generator generates and modulates electrosurgical energy which, in turn, is applied to the tissue by an electrosurgical instrument. Electrosurgical instruments may be either monopolar or bipolar and may be configured for open or endoscopic procedures.

Electrosurgical instruments may be implemented to ablate, seal, cauterize, coagulate, and/or desiccate tissue and, if needed, cut and/or section tissue. Typically, cutting and/or sectioning tissue is performed with a knife blade movable within a longitudinal slot located on or within one or more seal plates associated with one or more jaw members configured to receive a knife blade, or portion thereof. The longitudinal slot is normally located on or within the seal plate within a treatment zone (e.g., seal and/or coagulation zone) associated therewith. Consequently, the knife blade cuts and/or sections through the seal and/or coagulation zone during longitudinal translation of the knife blade through the longitudinal slot. In some instances, it is not desirable to cut through the zone of sealed or coagulated tissue, but rather to the left or right of the zone of sealed or coagulated tissue such as, for example, during a tonsillectomy and/or adenoidectomy procedure.

### SUMMARY OF THE DISCLOSURE

As noted above, after tissue is electrosurgically treated (e.g., sealed), it is sometimes desirable to cut tissue outside of the zone of treated tissue. With this purpose in mind, the present disclosure provides an electrosurgical apparatus that includes a housing having at least one shaft extending therefrom that operatively supports an end effector assembly at a distal end thereof. The end effector assembly includes first and second jaw members pivotably connected to each other and moveable from an open spaced apart position to a closed position. Each of the jaw members operatively couples to an electrically conductive seal plate. In an embodiment, one or both of the jaw members is configured to support one or more filaments thereon for selectively sectioning tissue. The electrically conductive seal plates and the filament each are adapted to connect to an electrical surgical energy source. In an embodiment, the electrosurgical apparatus is in operative communication with a control system having one or more control algorithms for independently controlling and/or monitoring the delivery of electrosurgical energy from the source of electrosurgical energy to the one or more filaments and the tissue sealing plate on each of the jaw members.

The present disclosure also provides a method for performing an electrosurgical procedure. The method includes the initial step of providing an electrosurgical apparatus that includes a pair of jaw members configured to grasp tissue therebetween. In embodiments, one or both of the jaw members may include one or more filaments. The method also includes the steps of: directing electrosurgical energy from an electrosurgical generator through tissue held between the jaw members; directing electrosurgical energy from the electrosurgical generator to one or more filaments in contact with or adjacent to tissue; and applying a force to tissue adjacent a portion of the effected tissue site such that the portion of effected tissue is detachable from the rest of the effected tissue.

The present disclosure further provides a system for performing an electrosurgical procedure. The system includes an electrosurgical apparatus adapted to connect to a source of electrosurgical energy. The electrosurgical apparatus includes a housing having at least one shaft extending therefrom that operatively supports an end effector assembly at a distal end thereof. The end effector assembly includes first and second jaw members pivotably connected to each other and moveable from an open spaced apart position to a closed position to grasp tissue. An electrically conductive tissue sealing plate operatively couples to each of the jaw members. In an embodiment, one or both of the jaw members is configured to support one or more filaments thereon for selectively sectioning tissue. The electrically conductive seal plates and the filament are adapted to connect to an electrical surgical energy source. In an embodiment, the electrosurgical apparatus is in operative communication with a control system. The control system includes one or more algorithms for independently controlling and monitoring the delivery of electrosurgical energy from the source of electrosurgical energy to the at least one filament and the tissue sealing plate on each of the jaw members.

### BRIEF DESCRIPTION OF THE DRAWING

Various embodiments of the present disclosure are described hereinbelow with references to the drawings, wherein:

FIG. 1 is a perspective view of an electrosurgical apparatus and electrosurgical generator adapted for use with an energy based sectioning (EBS) system intended for use during an electrosurgical procedure according to an embodiment of the present disclosure;

FIG. 2 is a block diagram illustrating components of the system of FIG. 1;

FIG. 3 is a schematic representation of an electrical configuration for connecting the electrosurgical apparatus to the electrosurgical generator depicted in FIG. 1;

FIG. 4A is an enlarged, side perspective view of an end effector assembly including a filament configuration intended for use with the EBS system of FIG. 1;

FIG. 4B is an enlarged view of the area of detail represented by 4B depicted in FIG. 4A;

FIGS. 5A-5C are enlarged, front perspective views of various filament configurations suitable for use with the end effector assembly of FIG. 4A;

FIGS. 6A-6B illustrate the electrosurgical apparatus depicted in FIG. 1 in use;

FIG. 7 is an enlarged, side view of an end effector assembly including a filament configuration intended for use with the EBS system of FIG. 1 according to another embodiment of the present disclosure; and

FIG. 8 is a flowchart of a method for performing an electrosurgical procedure according to an embodiment of the present disclosure.

### DETAILED DESCRIPTION

Detailed embodiments of the present disclosure are disclosed herein; however, it is to be understood that the disclosed embodiments are merely exemplary of the disclosure, which may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The present disclosure includes an electrosurgical apparatus that is adapted to connect to an electrosurgical generator that includes a control system configured for energy based sectioning (EBS).

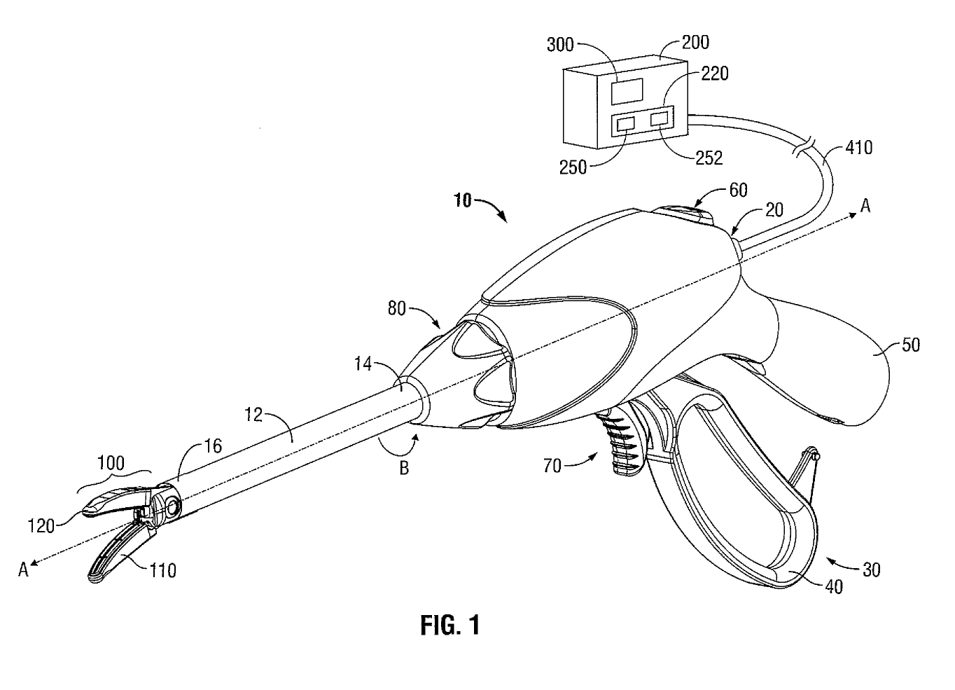

With reference to FIG. 1 an illustrative embodiment of an electrosurgical generator 200 (generator 200) is shown. Generator 200 is operatively and selectively connected to bipolar forceps 10 for performing an electrosurgical procedure. As noted above, an electrosurgical procedure may include sealing, cutting, coagulating, desiccating, and fulgurating tissue all of which may employ RF energy. Generator 200 may be configured for monopolar and/or bipolar modes of operation. Generator 200 includes all necessary components, parts, and/or members needed for a control system 300 (system 300) to function as intended. Generator 200 generates electrosurgical energy, which may be RF (radio frequency), microwave, ultrasound, infrared, ultraviolet, laser, thermal energy or other electrosurgical energy. An electrosurgical module 220 generates RF energy and includes a power supply 250 for generating energy and an output stage 252 which modulates the energy that is provided to the delivery device(s), such as an end effector assembly 100, for delivery of the modulated energy to a patient. Power supply 250 may be a high voltage DC or AC power supply for producing electrosurgical current, where control signals generated by the system 300 adjust parameters of the voltage and current output, such as magnitude and frequency. The output stage 252 may modulate the output energy (e.g., via a waveform generator) based on signals generated by the system 300 to adjust waveform parameters, e.g., waveform shape, pulse width, duty cycle, crest factor, and/or repetition rate. System 300 may be coupled to the generator module 220 by connections that may include wired and/or wireless connections for providing the control signals to the generator module 220.

With continued reference to FIG. 1, a system 300 for performing an electrosurgical procedure (e.g., RF tissue procedure) is shown. System 300 is configured to, among other things, analyze parameters such as, for example, power, tissue and filament temperature, current, voltage, power, impedance, etc., such that a proper tissue effect can be achieved.

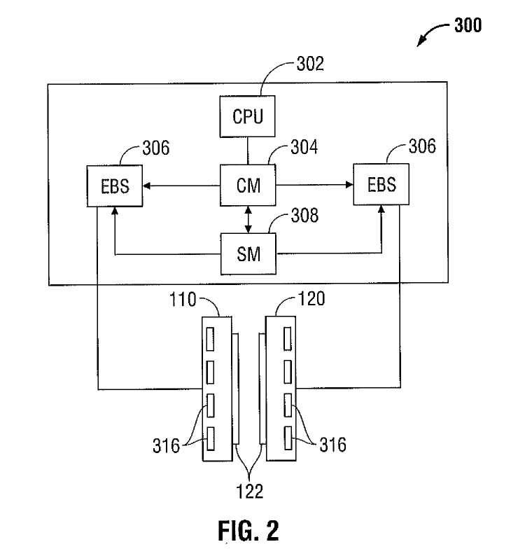

With reference to FIG. 2, system 300 includes one or more processors 302 in operative communication with a control module 304 executable on the processor 302, and is configured to, among other things, quantify electrical and thermal parameters during tissue sectioning such that when a threshold value for electrical and thermal parameters is met, the control system 300 provides a signal to a user to apply a force to tissue. Control module 304 instructs one or more modules (e.g., an EBS module 306) to transmit electrosurgical energy, which may be in the form of a wave or signal/pulse, via one or more cables (e.g., cable 410) to one or both of the seal plates 118, 128 and/or one or more filaments 122. Electrosurgical energy may be transmitted to the seal plates 118, 128 and the filaments 122 simultaneously or consecutively.

The control module 304 processes information and/or signals (e.g., tissue and/or filament temperature data from sensors 316) input to the processor 302 and generates control signals for modulating the electrosurgical energy in accordance with the input information and/or signals. Information may include pre-surgical data (e.g., tissue and/or filament temperature threshold values) entered prior to the electrosurgical procedure or information entered and/or obtained during the electrosurgical procedure through one or more modules (e.g., EBS module 306) and/or other suitable device(s). The information may include requests, instructions, ideal mapping(s) (e.g., look-up-tables, continuous mappings, etc.), sensed information and/or mode selection.

The control module 304 regulates the generator 200 (e.g., the power supply 250 and/or the output stage 252) which adjusts various parameters of the electrosurgical energy delivered to the patient (via one or both of the seal plates and/or one or more filaments) during the electrosurgical procedure. Parameters of the delivered electrosurgical energy that may be regulated include voltage, current, resistance, intensity, power, frequency, amplitude, and/or waveform parameters, e.g., waveform shape, pulse width, duty cycle, crest factor, and/or repetition rate of the output and/or effective energy.

The control module 304 includes software instructions executable by the processor 302 for processing algorithms and/or data received by sensors 316, and for outputting control signals to the generator module 220 and/or other modules. The software instructions may be stored in a storage medium such as a memory internal to the processor 302 and/or a memory accessible by the processor 302, such as an external memory, e.g., an external hard drive, floppy diskette, CD-ROM, etc.

In embodiments, an audio or visual feedback monitor or indicator (not explicitly shown) may be employed to convey information to the surgeon regarding the status of a component of the electrosurgical system or the electrosurgical procedure. Control signals provided to the generator module 220 are determined by processing (e.g., performing algorithms), which may include using information and/or signals provided by sensors 316.

The control module 304 regulates the electrosurgical energy in response to feedback information, e.g., information related to tissue condition at or proximate the surgical site. Processing of the feedback information may include determining: changes in the feedback information; rate of change of the feedback information; and/or relativity of the feedback information to corresponding values sensed prior to starting the procedure (pre-surgical values) in accordance with the mode, control variable(s) and ideal curve(s) selected. The control module 304 then sends control signals to the generator module 220 such as for regulating the power supply 250 and/or the output stage 252.

Regulation of certain parameters of the electrosurgical energy may be based on a tissue response such as recognition of when a proper seal is achieved and/or when a predetermined threshold temperature value is achieved. Recognition of the event may automatically switch the generator 200 to a different mode of operation (e.g., EBS mode or “RF output mode”) and subsequently switch the generator 200 back to an original mode after the event has occurred. In embodiments, recognition of the event may automatically switch the generator 200 to a different mode of operation and subsequently shutoff the generator 200.

EBS module 306 (shown as two modules for illustrative purposes) may be digital and/or analog circuitry that can receive instructions from and provide status to a processor 302 (via, for example, a digital-to-analog or analog-to-digital converter). EBS module 306 is also coupled to control module 304 to receive one or more electrosurgical energy waves at a frequency and amplitude specified by the processor 302, and/or transmit the electrosurgical energy waves along the cable 410 to one or both of the seal plates, one or more filaments 122 and/or sensors 316. EBS module 306 can also amplify, filter, and digitally sample return signals received by sensors 316 and transmitted along cable 410.

A sensor module 308 senses electromagnetic, electrical, and/or physical parameters or properties at the operating site and communicates with the control module 304 and/or EBS module 306 to regulate the output electrosurgical energy. The sensor module 308 may be configured to measure, i.e., “sense”, various electromagnetic, electrical, physical, and/or electromechanical conditions, such as at or proximate the operating site, including: tissue impedance, tissue temperature, and so on. For example, sensors of the sensor module 308 may include sensors 316, such as, for example, optical sensor(s), proximity sensor(s), pressure sensor(s), tissue moisture sensor(s), temperature sensor(s), and/or real-time and RMS current and voltage sensing systems. The sensor module 308 measures one or more of these conditions continuously or in real-time such that the control module 304 can continually modulate the electrosurgical output in real-time.

In embodiments, sensors 316 may include a smart sensor assembly (e.g., a smart sensor, smart circuit, computer, and/or feedback loop, etc. (not explicitly shown)). For example, the smart sensor may include a feedback loop which indicates when a tissue seal is complete based upon one or more of the following parameters: tissue temperature, tissue impedance at the seal, change in impedance of the tissue over time and/or changes in the power or current applied to the tissue over time. An audible or visual feedback monitor may be employed to convey information to the surgeon regarding the overall seal quality or the completion of an effective tissue seal.

With reference again to FIG. 1, electrosurgical apparatus 10 can be any type of electrosurgical apparatus known in the available art, including but not limited to electrosurgical apparatuses that can grasp and/or perform any of the above mentioned electrosurgical procedures. One type of electrosurgical apparatus 10 may include bipolar forceps as disclosed in United States Patent Publication No. 2007/0173814 entitled “Vessel Sealer and Divider For Large Tissue Structures”. A brief discussion of bipolar forceps 10 and components, parts, and members associated therewith is included herein to provide further detail and to aid in the understanding of the present disclosure.

With continued reference to FIG. 1, bipolar forceps 10 is shown for use with various electrosurgical procedures and generally includes a housing 20, a handle assembly 30, a rotating assembly 80, a trigger assembly 70, a shaft 12, a drive assembly (not explicitly shown), and an end effector assembly 100, which mutually cooperate to grasp, seal and divide large tubular vessels and large vascular tissues. Although the majority of the figure drawings depict a bipolar forceps 10 for use in connection with endoscopic surgical procedures, the present disclosure may be used for more traditional open surgical procedures.

Shaft 12 has a distal end 16 dimensioned to mechanically engage the end effector assembly 100 and a proximal end 14 which mechanically engages the housing 20. In the drawings and in the descriptions which follow, the term “proximal,” as is traditional, will refer to the end of the forceps 10 which is closer to the user, while the term “distal” will refer to the end which is farther from the user.

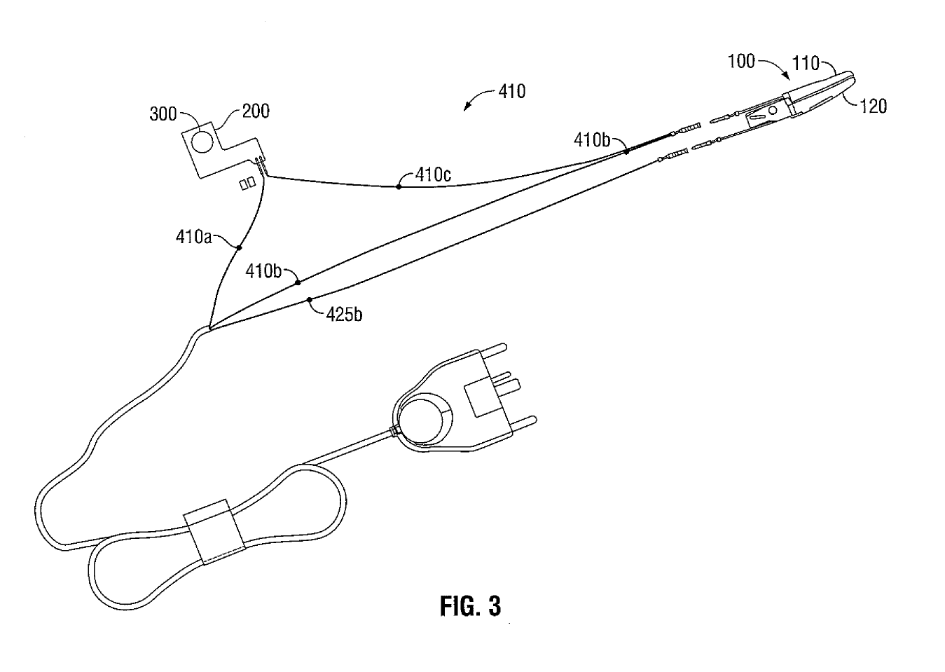

Forceps 10 includes an electrosurgical cable 410 that connects the forceps 10 to a source of electrosurgical energy, e.g., generator 200, shown schematically in FIG. 1. As shown in FIG. 3, cable 410 is internally divided into cable leads 410a, 410b and 425b which are designed to transmit electrical potentials through their respective feed paths through the forceps 10 to the end effector assembly 100.

For a more detailed description of handle assembly 30, movable handle 40, rotating assembly 80, electrosurgical cable 410 (including line-feed configurations and/or connections), and the drive assembly reference is made to commonly owned Patent Publication No., 2003-0229344, filed on Feb. 20, 2003, entitled VESSEL SEALER AND DIVIDER AND METHOD OF MANUFACTURING THE SAME.

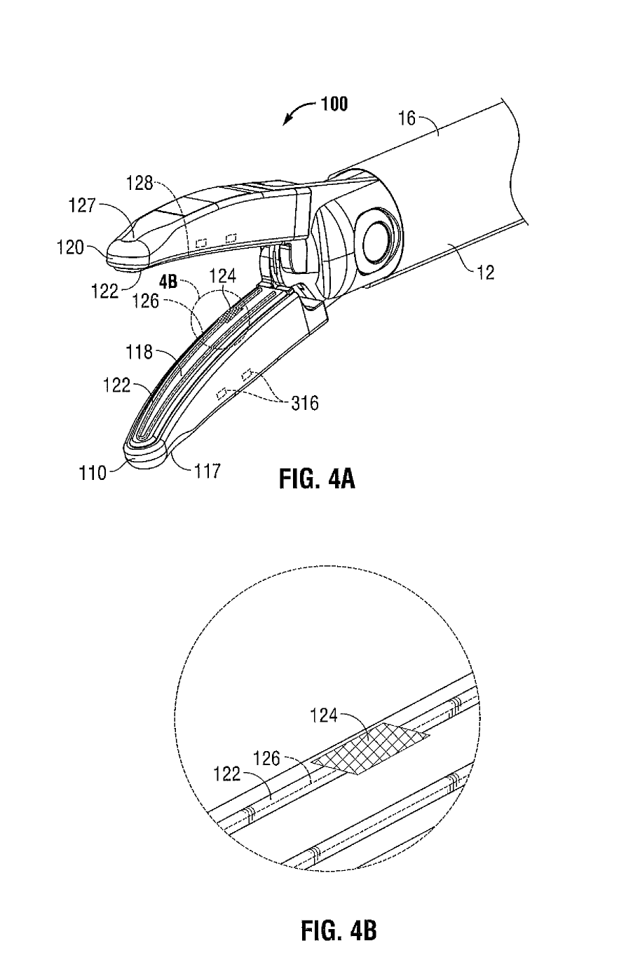

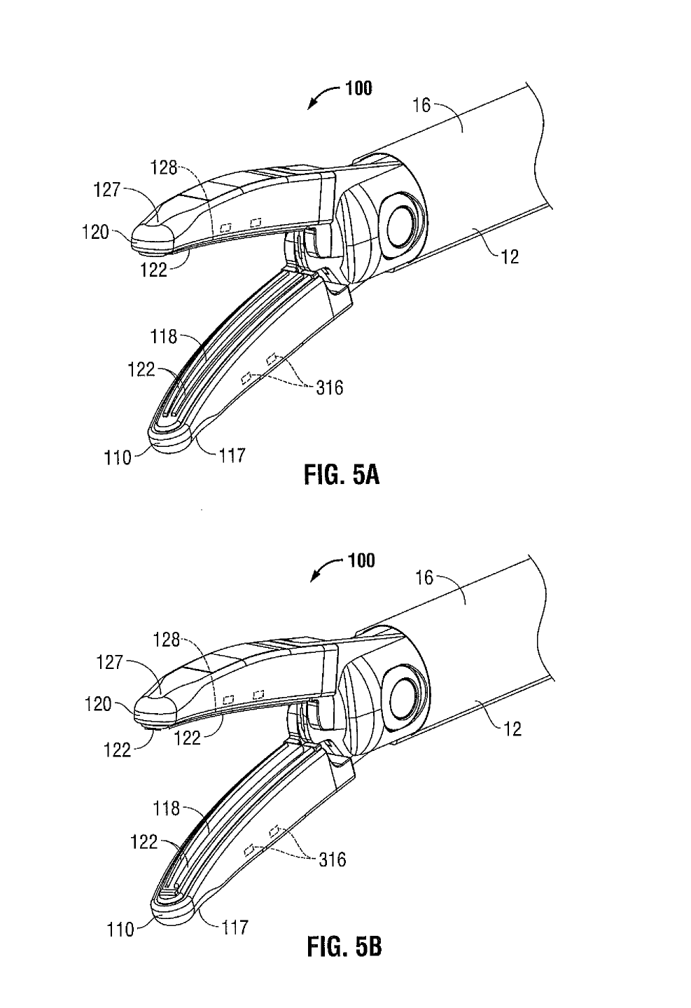

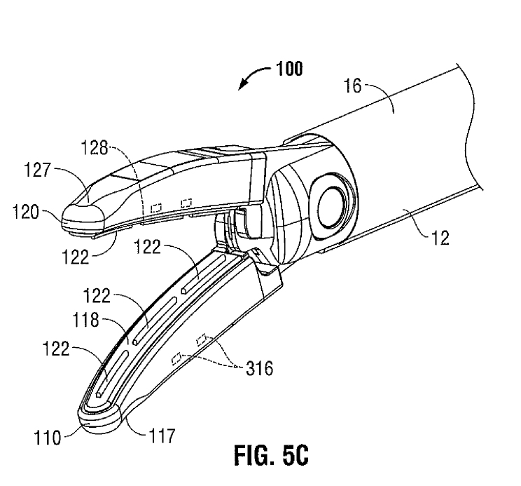

With reference now to FIGS. 4A, 5A-5C, and initially with reference to FIG. 4A, end effector assembly 100 is shown attached at the distal end 16 of shaft 12 and includes a pair of opposing jaw members 110 and 120. As noted above, movable handle 40 of handle assembly 30 operatively couples to a drive assembly which, together, mechanically cooperate to impart movement of the jaw members 110 and 120 from an open position wherein the jaw members 110 and 120 are disposed in spaced relation relative to one another, to a clamping or closed position wherein the jaw members 110 and 120 cooperate to grasp tissue therebetween.

Jaw members 110 and 120 are generally symmetrical and include similar component features which cooperate to effect the sealing and dividing of tissue. As a result, and unless otherwise noted, only jaw member 110 and the operative features associated therewith are described in detail herein, but as can be appreciated many of these features, if not all, apply to equally jaw member 120 as well.

Jaw member 110 includes an insulative jaw housing 117 and an electrically conductive seal plate 118 (seal plate 118). Insulator 117 is configured to securely engage the electrically conductive seal plate 118. Seal plate 118 may be manufactured from stamped steel. This may be accomplished by stamping, by overmolding, by overmolding a stamped electrically conductive sealing plate and/or by overmolding a metal injection molded seal plate. All of these manufacturing techniques produce an electrode having a seal plate 118 that is substantially surrounded by the insulating substrate. Within the purview of the present disclosure, jaw member 110 may include a jaw housing 117 that is integrally formed with a seal plate 118.

Jaw member 120 includes a similar structure having an outer insulative housing 127 that is overmolded (to capture seal plate 128).

End effector assembly 100 is configured for energy based sectioning (EBS). To this end, end effector assembly 100 is provided with one or more electrodes or filaments 122. Filament 122 may be configured to operate in monopolar or bipolar modes of operation, and may operate alone or in conjunction with control system 300 (mentioned and described above). With this purpose in mind, filament 122 is in operative communication with one or more sensors 316 operatively connected to one or more modules of control system 300 by way of one or more optical fibers or a cable (e.g., cable 410).

Filament 122 functions to convert electrosurgical energy into thermal energy such that tissue in contact therewith (or adjacent thereto) may be heated and subsequently cut or severed. With this purpose in mind, filament 122 may manufactured from any suitable material capable of converting electrosurgical energy into thermal energy and/or capable of being heated, including but not limited to metal, metal alloy, ceramic and the like. Metal and/or metal alloy suitable for the manufacture of filament 122 may include Tungsten, or derivatives thereof. Ceramic suitable for the manufacture of filament 122 may include those of the non-crystalline (e.g., glass-ceramic) or crystalline type.

Filament 122 is configured to contact tissue during or after application of electrosurgical energy that is intended to treat tissue (e.g., seal tissue). To this end, filament 122 is disposed at predetermined locations on one or both of the jaw members 110, 120, see FIG. 4A for example. As shown, filament 122 extends from and along seal plate 118 of jaw member 110. Filaments 122 disposed on the jaw members 110, 120 may be in vertical registration with each other.

The top portion of filament 122 may have any suitable geometric configuration. For example, FIG. 4A illustrates filament 122 having a top portion that is curved, while FIGS. 5A and 5B illustrate, respectively, one or more filaments 122 each having top portions that are flat and one or more filaments 122 each having top portions that are curved, flat, and pointed.

To prevent short-circuiting from occurring between the filament 122 and the seal plate (e.g., seal plate 118) from which it extends or is adjacent thereto, filament 122 is provided with an insulative material 126, as best seen in FIG. 4B. The insulative material 126 may be disposed between the portion of the filament 122 that extends from or that is adjacent to the seal plate. Alternatively, or in addition thereto, the portion of the filament 122 that extends from or that is adjacent to the seal plate may be made from a non-conductive material. In embodiments, one or more filaments 122 may have portions that are insulated and/or separated from each other (see FIGS. 5A-5C, for example).

Filament 122 may be active prior, during, or subsequent to the application of electrosurgical energy used for performing an electrosurgical procedure (e.g., sealing). Filament 122, or portions thereof, may be activated and/or controlled individually and/or collectively.

In embodiments, filament 122 may be coated with a conductive non-stick material 124, such as, for example, a conductive non-stick mesh, as best seen in FIG. 4B. Filament 122 coated with a conductive non-stick material 124 or conductive non-stick mesh may prevent and/or impede sticking and/or charring of tissue during the application of electrosurgical energy for performing the electrosurgical procedure or EBS.

One or both of the jaw members 110, 120 may include one or more sensors 316. Sensors 316 are placed at predetermined locations on, in, or along surfaces of the jaw members 110, 120 (FIGS. 4A and 5A-5C). In embodiments, end effector assembly 100 and/or jaw members 110 and 120 may have sensors 316 placed near a proximal end and/or near a distal end of jaw members 110 and 120, as well as along the length of jaw members 110 and 120.

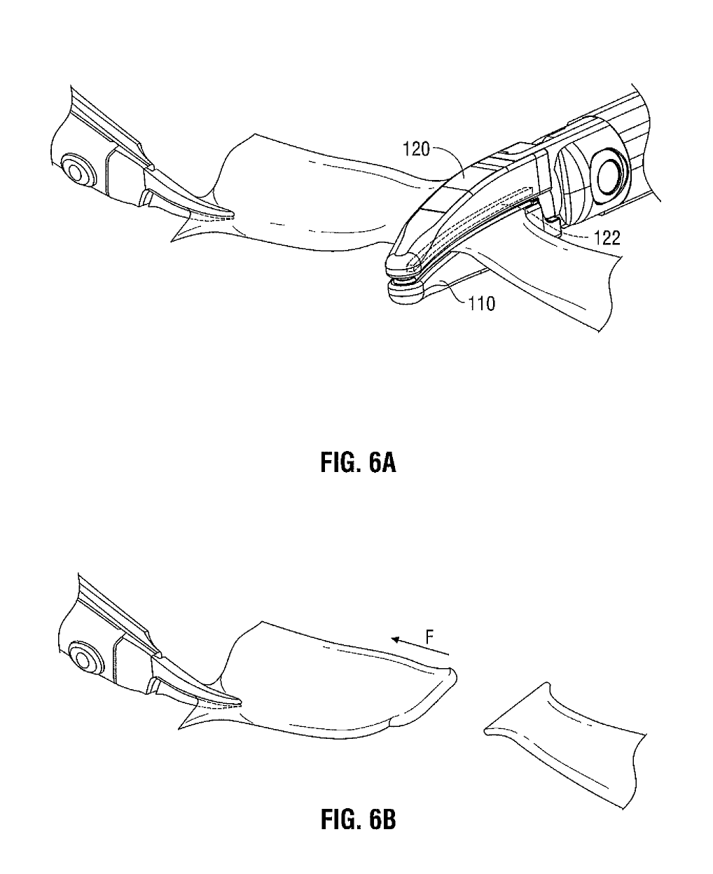

With reference now to FIGS. 6A and 6B, operation of bipolar forceps 10 under the control of system 300 is now described. For illustrative purposes, EBS is described subsequent to the application of electrosurgical energy for achieving a desired tissue effect (e.g., tissue sealing). Processor 302 instructs EBS module 306 to generate electrosurgical energy in response to the processor instructions, the EBS module 306 can access a pulse rate frequency clock associated with a time source (not explicitly shown) to form an electrosurgical pulse/signal exhibiting the attributes (e.g., amplitude and frequency) specified by the processor 302 and can transmit such pulse/signal on one or more cables (e.g., cable 410) to filament 122 and/or sensors 316. In another embodiment, the processor does not specify attributes of the electrosurgical pulse/signal, but rather instructs/triggers other circuitry to form the electrosurgical pulse/signal and/or performs timing measurements on signals conditioned and/or filtered by other circuitry.

The transmitted electrosurgical pulse/signal travels along cable 410 to one or more filaments 122 that is/are in contact with, and/or otherwise adjacent to tissue. Filament 122 converts the electrosurgical energy to thermal energy and heats the tissue in contact therewith or adjacent thereto. Data, such as, for example, temperature, pressure, impedance and so forth is sensed by sensors 316 and transmitted to and sampled by the EBS module 306 and/or sensor module 224.

The data can be processed by the processor 302 and/or EBS module 306 to determine, for example, when a tissue and/or filament threshold temperature has been achieved. The processor 302 can subsequently transmit and/or otherwise communicate the data to the control module 304 such that output power from generator 200 may be adjusted accordingly. The processor 302 can also subsequently transmit and/or otherwise communicate the data to a local digital data processing device, a remote digital data processing device, an LED display, a computer program, and/or to any other type of entity (none of which being explicitly shown) capable of receiving the data.

Upon reaching a desired tissue and/or filament 122 threshold temperature, control system 300 may indicate (by way of an audio or visual feedback monitor or indicator, previously mentioned and described above) to a user that tissue is ready for sectioning. A user may then grasp tissue (for example, with a surgical implement or bipolar forceps 10) adjacent to the operating site and outside the seal zone (FIG. 6A) and apply a pulling force “F” generally normal and along the same plane as the sectioning line which facilitates the separation of tissue (FIG. 6B). Application of the pulling force “F” separates the unwanted tissue from the operating site with minimal impact on the seal zone. The remaining tissue at the operating site is effectively sealed and the separated tissue may be easily discarded.

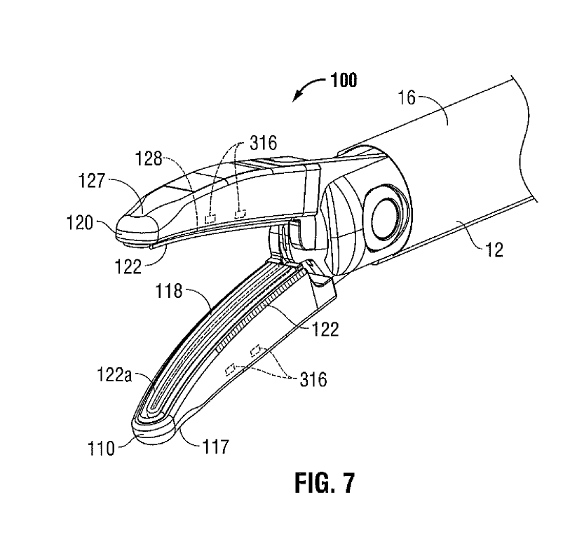

From the foregoing and with reference to the various figure drawings, those skilled in the art will appreciate that certain modifications can also be made to the present disclosure without departing from the scope of the same. For example, as best seen in FIG. 7, it may be preferable to include a channel or cavity 122a (shown phantomly) on one or both of the seal plates (e.g., seal plate 118) that is in vertical registration with a filament 122 on an opposing seal surface (e.g., seal plate 128). Here, the cavity 122a and the filament 122 are configured to matingly engage with each other when the jaw members are in a closed configuration such that effective heating of tissue at the tissue site may be achieved. As can be appreciated by one skilled in the art, a filament 122 of a given structure configured to matingly engage with a corresponding cavity 122a may allow the filament 122 to contact a greater tissue area which, in turn, may enable a user to heat more tissue for a given EBS procedure.

While a majority of the drawings depict a filament 122 that is disposed on one or both of the seal plates of one or both of the jaw members 110, 120, it is within the purview of the present disclosure to have one or more filaments 122 disposed on and/or along an outside and/or inside edge of one or both of the jaw members 110, 120, or any combination thereof. For example, filament 122 may extend partially along an outside edge of jaw member 110 (see FIG. 7, for example). Alternatively, filament 122 may extend along the entire length of a periphery of jaw member 110. In either instance, filament 122 may be configured as described above and/or may include the same, similar and/or different structures to facilitate separating tissue.

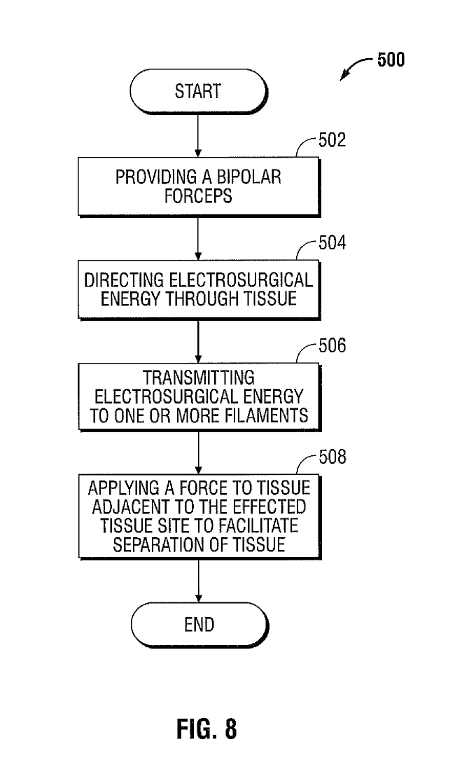

FIG. 8 shows a method 500 for performing an electrosurgical procedure. At step 502, an electrosurgical apparatus including a pair of jaw members configured to grasp tissue therebetween and including one or more filaments is provided. At step 504, electrosurgical energy from an electrosurgical generator is directed through tissue held between the jaw members. At step 506, electrosurgical energy from the electrosurgical generator is transmitted to one or more filaments in contact with or adjacent to tissue such that tissue may be severed. And at step 508, a force is applied to tissue adjacent the effected tissue site generally in a normal or transverse direction to facilitate separation of the tissue.

In embodiments, the step of delivering electrosurgical energy to the at least one filament may include the step of system 300 quantifying one of electrical and thermal parameter associated with tissue and the filament.

In embodiments, the step of applying a force may include the step of applying the force simultaneously with delivering electrosurgical energy from the source of electrosurgical energy to the at least one filament.

In embodiments, the step of applying a force may include the step of applying the force consecutively after audible or visible indication (e.g., an LED located on generator 200 displays “Apply Pulling Force”).

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An electrosurgical system, comprising:
an electrosurgical apparatus having an end effector assembly including first and second jaw members pivotably connected to each other and moveable from an open spaced apart position to a closed position to grasp tissue; and
an electrically conductive tissue sealing plate operatively coupled to each of the jaw members, at least one of the jaw members configured to support at least one filament thereon configured for selectively sectioning tissue, the electrically conductive seal plates and the filament adapted to connect to an electrical surgical energy source;
wherein the electrosurgical apparatus is in operative communication with a control system having at least one algorithm for at least one of independently controlling and monitoring the delivery of electrosurgical energy from the source of electrosurgical energy to the at least one filament and the tissue sealing plate on each of the jaw members.

2. An electrosurgical system according to claim 1, wherein the at least filament is located along a periphery of the tissue sealing plate.

3. An electrosurgical system according to claim 1, wherein the at least filament is located on an inside edge of the at least one of the jaw members.

4. An electrosurgical system according to claim 1, wherein the at least filament is coated with a conductive non-stick material.

5. An electrosurgical system according to claim 4, wherein the conductive non-stick material is a conductive mesh.

6. An electrosurgical system according to claim 1, wherein the control system is configured to delivery electrosurgical energy to the at least one filament and at least one of the tissue sealing plates simultaneously.

7. An electrosurgical system according to claim 1, wherein the control system is configured to delivery electrosurgical energy to the at least one filament and at least one of the tissue sealing plate consecutively.

8. An electrosurgical system according to claim 2, wherein the at least one filament is electrically insulated from the electrically conductive sealing plates.

9. An electrosurgical system according to claim 1, wherein filament has a generally curved top portion.

10. An electrosurgical system according to claim 1, wherein filament has a relatively flat top portion.

11. An electrosurgical system according to claim 1, wherein filament has a pointed top portion.

12. An electrosurgical system according to claim 1, at least one of the jaw members includes at least one filament and an opposing jaw member includes at least one corresponding cavity in vertical registration with the at least one filament and configured to receive at least a portion of the at least one filament.

13. An electrosurgical system according to claim 1, wherein the control system quantifies one of electrical and thermal parameters during tissue sectioning such that when a threshold value for the one of electrical and thermal parameters is met the control system provides a signal to a user to apply a force to tissue.

14. A method for performing an electrosurgical procedure the method comprising:
providing an electrosurgical system, comprising:
an electrosurgical apparatus having an end effector assembly including first and second jaw members;
a tissue sealing plate disposed on each of the jaw members, at least one of the jaw members configured to support at least one filament thereon; and
wherein the electrosurgical apparatus is in operative communication with a control system having at least one algorithm for at least one of independently controlling and monitoring the delivery of electrosurgical energy from a source of electrosurgical energy to the at least one filament and the tissue sealing plate on each of the jaw members;
delivering electrosurgical energy from the source of electrosurgical energy to each of the seal plates until a desired tissue effect is achieved;
delivering electrosurgical energy from the source of electrosurgical energy to the at least one filament; and
applying a force adjacent to at least a portion of the effected tissue such that the at least a portion of the effected tissue is detachable from the rest of the effected tissue.

15. A method according to claim 14, wherein the steps of delivering electrosurgical energy to each of the seal plates and delivering electrosurgical energy to the at least one filament are done simultaneously.

16. A method according to claim 14, wherein the steps of delivering electrosurgical energy to each of the seal plates and delivering electrosurgical energy to the at least one filament are done consecutively.

17. A method according to claim 14, wherein the electrosurgical apparatus includes at least one filament thereon configured for sectioning tissue, the filament including an conductive mesh.

18. A method according to claim 14, wherein the electrosurgical apparatus includes at least one filament on at least one of the seal plates and a corresponding cavity in vertical registration with the at least one filament on the other seal plate.

19. A method according to claim 14, wherein the step of delivering electrosurgical energy to the at least one filament includes the step of quantifying one of electrical and thermal parameter associated with tissue and the filament.

20. A system for performing an electrosurgical procedure, comprising:
an electrosurgical apparatus adapted to connect to a source of electrosurgical energy, the electrosurgical apparatus including a housing having at least one shaft extending therefrom that operatively supports an end effector assembly at a distal end thereof, the end effector assembly including first and second jaw members pivotably connected to each other and moveable from an open spaced apart position to a closed position to grasp tissue; and
an electrically conductive tissue sealing plate operatively coupled to each of the jaw members, at least one of the jaw members configured to support at least one filament thereon configured for selectively sectioning tissue, the electrically conductive tissue sealing plates and the filament adapted to connect to an electrical surgical energy source;
wherein the electrosurgical apparatus is in operative communication with a control system having at least one algorithm for independently controlling and monitoring the delivery of electrosurgical energy from the source of electrosurgical energy to the at least one filament and the tissue sealing plate on each of the jaw members.
